(19) United States
(12) Patent Application Publication
(10) Pub. No.: US 2009/0054538 A1
(43) Pub. Date: Feb. 26, 2009

(54) Chemical production processes, systems, and catalyst compositions

(76) Inventors: Thomas H. Peterson, Midland, MI (US); Alan H. Zacher, Pasco, WA (US); Michel J. Gray, Pasco, WA (US); James F. White, Richland, WA (US); Johnathon E. Holladay, Kennewick, WA (US); Todd A. Werpy, West Richland, WA (US)

Correspondence Address: WELLS ST. JOHN P.S., 601 WEST FIRST AVENUE, SUITE 1300, SPOKANE, WA, 99201-3828, US

(21) Appl. No.: 11/895,592

(22) Filed: Aug. 24, 2007

(51) Int. Cl. C07C 27/22

(52) U.S. Cl. 518/705; 422/129; 502/246; 502/254; 502/305; 502/311; 518/714

(57) ABSTRACT

Chemical production processes are provided than can include exposing a reactant composition to a catalyst composition to form a product composition, with the reactant composition including a multihydric alcohol compound and product composition including a carbonyl compound. The catalyst composition can include one or more elements of groups 5 and 6 of the periodic table of elements. Catalyst compositions are provided that can include one or more of niobia, hydrated niobia, tungstic acid, phosphotungstic acid, and phosphomolybdic acid.

### RELATED PATENT DATA

This application is a continuation in part of U.S. patent applications: Ser. No. 11,895,362, entitled Chemical Production Processes, Systems, and Catalyst Compositions by Peterson et. al. which was filed on Aug. 24, 2007; Ser. No. 11,895,593, entitled Chemical Production Processes, Systems, and Catalyst Compositions by Peterson et. al. which was filed on Aug. 24, 2007; Ser. No. 11,895,414, entitled Chemical Production Processes, Systems, and Catalyst Compositions by Peterson et. al. which was filed on Aug. 24, 2007; the entirety of all are incorporated by reference herein.

### TECHNICAL FIELD

The present disclosure relates to chemical production processes, systems, and catalyst compositions.

### BACKGROUND OF THE DISCLOSURE

For nearly a century, scientists have struggled with the efficient dehydration of multihydric alcohol compounds. For example, the dehydration of glycerol to acrolein, acetol, and glycerol oligomers was first reported by Nef in 1904. When compositions were heated to temperatures between 430° C. and 450° C., carbonaceous materials and a distillate were produced that contained acrolein, acetol, water, and formaldehyde among other products. Over the next 100 years, occasional reports of catalyzed conversions of glycerol had been communicated targeting conversion to acrolein and acetol directly. As an example, the condensation of glycerol to di-, tri-, and oligoglycerol ethers had been effected with basic catalysts. However, when acidic catalysts were employed, acrolein is formed as a major by-product. As another example, acrolein has also been reported as a product of castor oil hydrolysis and cracking. Conversion of multihydric alcohol compounds has been performed in the temperature range of 250° C. to 400° C., utilizing phosphate or sulfate acid or acid salt as a catalyst. However, clays, zeolites, CO₂ or autoionization of supercritical H₂O has been shown to effect dehydration with the yields of acrolein rarely exceeding 70%.

While examples of glycerol to acrolein transformation do exist, they are relatively few in number. As recently as 1994, U.S., Japanese, and European patents have been awarded describing the conversion of glycerol to acrolein and acrolein hydrogenation to a mixture of isomeric propanediols. In 1998, platinum bisphosphine complexes were used in the presence of strong acids and syn gas to carry out the conversion of glycerol to acrolein in 80% yield. More recently, glycerol dehydration in subcritical water catalyzed by ZnSO₄ in a staged reactor process has been utilized to convert glycerol to acrolein and acrylic acid. To date, processes having high selectivity and commercial viability are still not known for the conversion of glycerol to acrolein.

### SUMMARY OF THE DISCLOSURE

Chemical production processes are provided that can include exposing a reactant composition to a catalyst composition to form a product composition, with the reactant composition including a multihydric alcohol compound and product composition including a carbonyl compound. The catalyst composition can include one or more elements of groups 5 and 6 of the periodic table of elements.

Catalyst compositions are provided that can include one or more of niobia, hydrated niobia, tungstic acid, phosphotungstic acid, and phosphomolybdic acid.

Chemical production systems are provided that can include a reactant reservoir coupled to a reactor with the reactor containing a catalyst comprising one or more elements of groups 5 and 6 of the periodic table of elements.

### BRIEF DESCRIPTION OF THE DRAWINGS

Preferred embodiments of the disclosure are described below with reference to the following accompanying drawings.

FIG. 1 is a chemical production system according to an embodiment of the disclosure.

FIG. 2 is a chemical production system according to another embodiment of the disclosure.

FIG. 3 is a plot of data acquired utilizing an embodiment of the disclosure.

FIG. 4 is a plot of data acquired utilizing an embodiment of the disclosure.

### DESCRIPTION

This disclosure is submitted in furtherance of the constitutional purposes of the U.S. Patent Laws “to promote the progress of science and useful arts” (Article 1, Section 8).

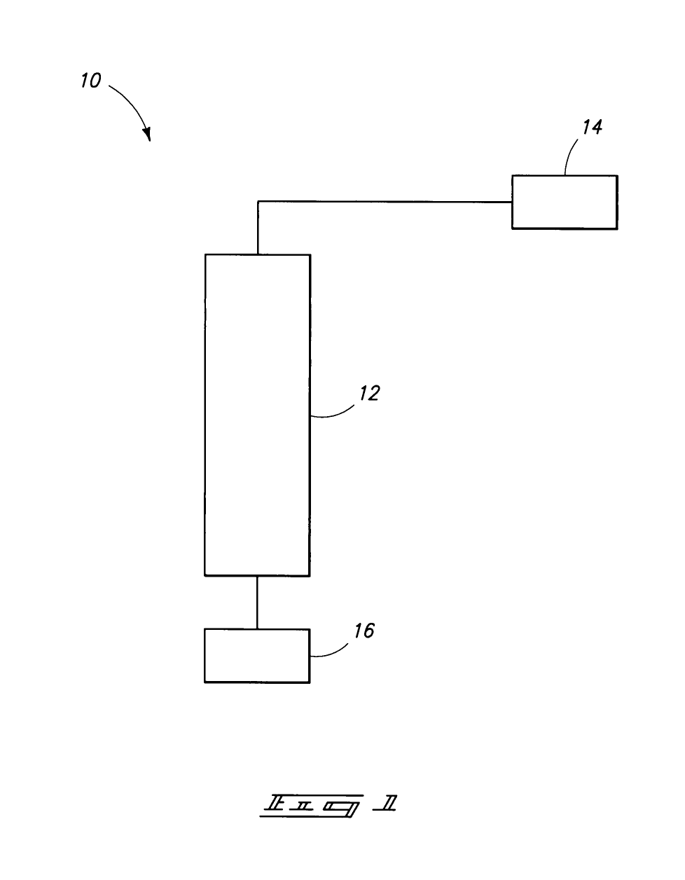

The chemical production processes of the present disclosure will be described with reference to FIGS. 1-4. Referring first to FIG. 1, a chemical production process system 10 is shown that includes a reactor 12 coupled to both a reactant reservoir 14 and a product reservoir 16. In accordance with the present disclosure, reactant reservoir 14 can be coupled to reactor 12 utilizing conduits that facilitate the flow of reactant from reactant reservoir 14 to reactor 12. This flow can be facilitated utilizing pressure differentials between reactant reservoir 14 and reactor 12. For example, these pressure differentials can be facilitated utilizing pumps to provide a pressure differential between reactant reservoir 14 and reactor 12. The reactant within reactant reservoir 14 can be a multihydric alcohol compound. An example multihydric alcohol compound can include the compound glycerol, which when dehydrated can result in a product composition that includes one or both of acrolein and/or acetol, for example.

Reactor 12 can include a housing that can be configured to house a catalyst and be utilized to facilitate the exposure of the reactant within reactant reservoir 14 to catalyst within reactor 12. The catalyst can be supported and/or unsupported catalyst, for example. Unsupported catalysts can be referred to as bulk catalysts. Reactor 12 can be jacketed or can be configured as a fluidized bed reactor, for example.

The product composition provided to product reservoir 16 can be a dehydration product of the multihydric alcohol compound such as a carbonyl compound. The pressure differential apparatus used to facilitate the transfer of reactant from reactant reservoir 14 can also be utilized to provide product from reactor 12 to product reservoir 16. In accordance with an example embodiment, system 10 can be configured to expose a multihydric alcohol compound such as glycerol from reservoir 14 to a catalyst composition within reactor 12 to form a product composition including one or both of acrolein and acetol.

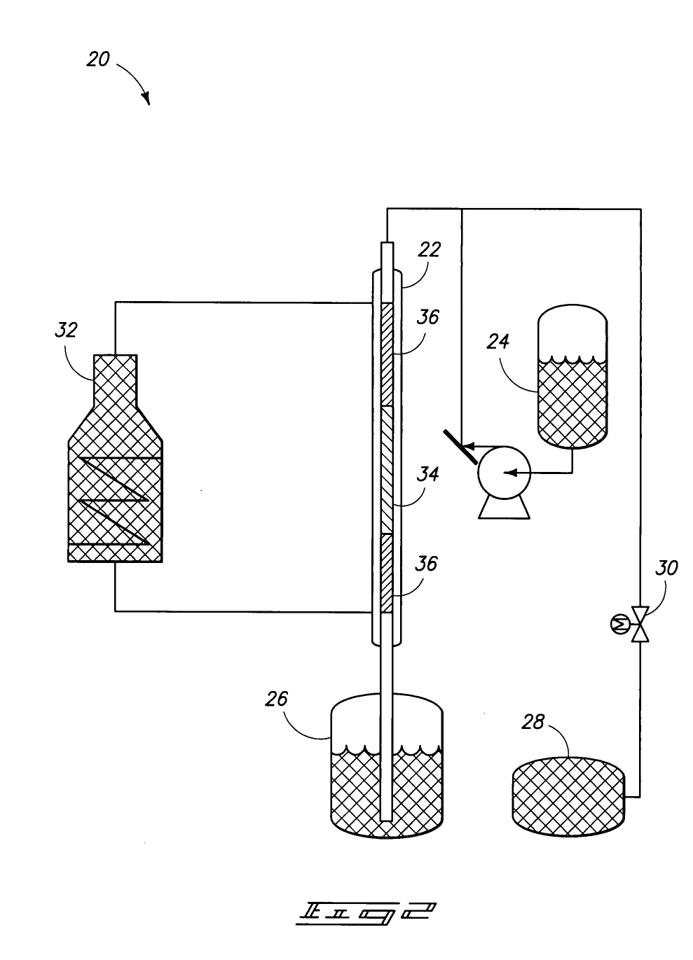

In accordance with another embodiment, FIG. 2 depicts a chemical production system 20 that includes a reactor 22 coupled to a reactant reservoir 24 as well as a product reservoir 26. Reactant of reactant reservoir 24 can be a multihydric alcohol compound, for example. To facilitate the flow of reactant from reactant reservoir 24 to reactor 22, a carrier composition 28 including a gas or liquid such as nitrogen is provided to a reactant reservoir conduit utilizing flow control 30. In accordance with another embodiment CO₂ can be utilized as the carrier composition 28. These solid support beds were also treated with CO₂ and reactant from reactor reservoir 24 can be combined with carrier composition 28 and provided to reactor 22.

Reactor 22 can be configured as an oil heated reactor utilizing an oil heater 32. Reactor 22 can be configured having a catalyst 34 supported by packing material 36. Catalyst 34 can include elements of group 5 and 6 of the periodic table of elements, for example such as the polyoxometallates of these elements. More particularly, catalyst 34 can include Nb, Mo, and/or W. Catalyst 34 can be hydrated or oxided. For example, catalyst 34 can be hydrated nobia. Catalyst 34 can include tungstic acid and/or phosphotungstic acid. Catalyst 34 can include phosphomolybdic acid. Catalyst 34 can be supported with a silica support. Prior to exposing reactant to catalyst 34, catalyst 34 can be exposed to carrier composition 28 such as CO₂.

Catalyst 34 can be prepared in a jar by placing 6 g of tungstic acid, for example, with a stir bar in the jar. The acid can be slurried with 20 mL of H₂O and approximately 15 mL of 15M aqueous ammonia (solution pH=12.5). Colloidal silica (58 g, 42 mL) can then be added to the jar with vigorous stirring resulting in precipitation. After stirring for 2 hours, the solution can be concentrated to dryness on a rotary evaporator with a bath temperature of 50° C. and operating at a pressure of 40 torr. The solid can be transferred to a porcelain crucible and calcined for 6 hours at 330° C. to provide a solid product. Subsequently, the solid product can be crushed and sieved to 45×100 mesh, for example.

Packing material 36 can include quartz wool. In accordance with one example, glycerol from reactant reservoir 24 can be provided with carrier composition 28 to reactor 22 and exposed to catalyst 34 such as solid support materials. A temperature within reactor 24 during exposure of reactant to these solid support materials is in the range of about 200° C. to about 500° C., more particularly in the range of from about 280° C. to about 320° C. Reactant can reside in reactor 24 for contact times of from about 250 to about 300 milliseconds.

In accordance with example implementations, the production process can include exposing the multihydric alcohol compound glycerol to catalyst 34 to form a product composition that includes both acrolein and acetol. For every mole of glycerol exposed to catalyst 34, at least about 0.1 moles of product composition can be formed and/or from about 0.1 to about 0.99 moles of product composition can be formed. Further, a mole ratio of acrolein to acetol within the product composition can be at least about 3:2 and/or from about 3:2 to about 10:1.

Hydrated forms of metal oxides of catalyst 34 can demonstrate acidic behavior, and the overall acidity can be controlled by the degree of hydration. In some cases, (niobia, for example) the degree of hydration can itself be controlled by the pre-treatment temperature of the oxide. Thus pretreatment can provide a vehicle for tuning the acidity of a material.

Pelleted niobia can be calcined for periods of 6 hours at temperatures of 200° C., 300° C., 400° C., and 500° C. The calcined materials can then be crushed and sieved to a size appropriate for the reactor and evaluated at temperatures between 280° C. and 320° C. employing contact times of 300 milliseconds or less. Results for catalysts, which are tabulated as averages of multiple runs, are shown in Tables 1 and 2 below.

TABLE 1
Performance of hydrated niobia for glycerol dehydration at 300° C.

| Calcination | | Carbon | | Acrolein | Acrolein | Acetol |
|---|---|---|---|---|---|---|
| Temperature | Sup- | Balance | Conv. | Yield | Selec- | Selec- |
| (° C.) | port | (%) | (%) | (%) | tivity | tivity |
| 200 | bulk | 56.70 | 85.88 | 34.85 | 0.41 | 0.09 |
| 300 | bulk | 66.10 | 89.48 | 47.39 | 0.530 | 0.022 |
| 400 | bulk | 50.52 | 95.43 | 38.94 | 0.41 | 0.07 |
| 400 | silica | 93.45 | 55.28 | 38.89 | 0.72 | 0.19 |
| 500 | bulk | 53.56 | 3.32 | 5.28 | 0.000 | 0.000 |

TABLE 2
Performance of hydrated niobia for glycerol dehydration at 320° C.

| Calcination | | Carbon | | Acrolein | | Acetol |
|---|---|---|---|---|---|---|
| Temperature | Sup- | Balance | Conv. | Yield | Acrolein | Selec- |
| (° C.) | port | (%) | (%) | (%) | Selectivity | tivity |
| 200 | bulk | 52.35 | 99.64 | 44.23 | 0.44 | 0.08 |
| 300 | bulk | 82.65 | 97.78 | 70.90 | 0.725 | 0.097 |
| 400 | bulk | 69.26 | 99.09 | 56.40 | 0.569 | 0.121 |
| 400 | silica | 88.98 | 75.02 | 50.58 | 0.67 | 0.18 |
| 500 | bulk | 46.95 | 22.65 | 14.25 | 0.629 | 0.236 |

The qualitative and quantitative data of Tables 1 and 2 above as well as all remaining data of the present application can be acquired utilizing gas and liquid chromatography techniques. For example, gas chromatographic analyses can be performed utilizing a Shimadzu GC-2010 Gas Chromatograph (GC) equipped with a Flame Ionization Detection (FID) operating at 280° C., and an AOC-20 autosampler, and employing GC Solutions Software. A DB-WAX (J & W Scientific) capillary column (30 m×0.32 mm I.D.×0.25 μm film thickness) can be employed utilizing helium as carrier gas at a 2.61 mL/min flow rate. Injections of 1 μL utilizing a 25:1 split ratio can be made with the injector port maintained at 250° C. Oven temperature programming can utilize an initial temperature of 40° C. with a hold for 5 minutes followed by a 10° C./min ramp to 245° C. and a hold at the final temperature for 4.5 minutes. Calibrations can be performed on a periodic or monthly basis using known standard solutions for glycerol, acrolein, and acetol. Calibrations can take place using a series of five standard solutions prepared by serial dilution to determine the linear response for each compound, and acceptance of each curve determined if the linear response had an R value of greater than 0.99.

Liquid chromatographic analyses can be carried out on a Waters LC system incorporating a Waters 515 pump, Waters 2410 Refractive Index Detector (RID), and a Waters 717plus Autosampler for sample introduction. Analyses can be performed utilizing Empower Pro Software. Separations of 10 μL injections can be effected on an Aminex HPX-87H Organic Acid Analysis column operated at 35° C. and employing a 0.005 M H₂SO₄ as the eluent with a flow rate of 0.55 mL/min. Total run times of 45 minutes were sufficient to elute all compounds of interest. Calibration curves can be prepared as described for GC calibrations and using the same set of standard solutions used for GC calibration.

Referring to product reservoir 26 of system 20, upon exiting reactor 22, product can be acquired by time collection of reactor 22 effluent in a known quantity of a chilled scrub solution containing 1 wt % n-BuOH with mass balances for a given reactor run determined by a ratio of collected effluent mass to expected mass based on feed rate and run time. For example, two small aliquots can be removed and diluted to concentrations appropriate for GC and LC analyses. The diluted samples can then be analyzed as described previously and wt % compositions determined from calibrated detector responses used to determine absolute compositions of the collected effluent. The use of known quantities of n-BuOH in the scrub solutions can permit a primary check of analytical sampling technique. Reported values for conversion, yield, selectivity, and carbon balance present averages of those values determined by both GC and LC analyses. Glycerol conversion can be calculated by the differences between calculated quantity of glycerol feed (based on feed rate and run time) and the quantity of glycerol collected in the reactor effluent and may be uncertain when mass balances are not satisfactory. Values exclude any experimental runs that did not provide mass balances between 90% and 100%. Product yields can be calculated by the ratio of quantity of product formed to the quantity of glycerol. Product selectivities can be calculated from the quantity of product formed divided by the quantity of glycerol converted. Carbon balances can be calculated from the sum of the molar quantities of glycerol, acrolein, and acetol components divided by the molar quantity of glycerol fed. Liquid Chromatographic techniques can permit the quantification of formic acid and acetic acid by-products. However, since their combined quantity rarely exceeded 3%, their presence was not included in carbon balance determination.

Referring to Tables 1 and 2, for catalyst calcined at 500° C., conversion can be reduced, consistent with reduced acidity. The differences between niobia samples calcined at 500° C. and lower temperatures can indicate that niobia rehydration to more acidic forms, even under hydrothermal conditions, may not occur. For runs at 300 and 320° C., the niobia samples calcined at 200° C. can provide low yields and carbon balances. Niobia deposition onto an inert support, (Tables 1 and 2, silica entry) can result in lower activity but increased selectivity. Both observations can be consistent with reduced total acidity in the prepared catalysts which lead to lower rates for both catalytic dehydration and catalyst-promoted product decomposition.

Commercial samples of tungstic and phosphotungstic acid can be dissolved in water and deposited onto colloidal silica to prepare catalyst 34 as well. The supported catalysts can be evaluated for glycerol dehydration at 300° C. and 320° C. employing contact times of 140 and 250 milliseconds. Data are tabulated below in tables 3 and 4.

TABLE 3
Performance of tungsten acids for glycerol dehydration at 300° C.

| | Con- | | | | | |
|---|---|---|---|---|---|---|
| Catalyst | tact | Carbon | | Acrolein | | Acetol |
| (20 wt % on | Time | Balance | Conv. | Yield | Acrolein | Selec- |
| SiO₂) | (sec) | (%) | (%) | (%) | Selectivity | tivity |
| H₂WO₄ | .014 | 95.87 | 41.1 | 33.43 | 0.813 | 0.086 |
| H₃O₄₄W₁₂P | | 94.49 | 35.65 | 28.12 | 0.785 | 0.086 |
| H₃Mo₁₂O₄₀P | | 32.38 | 38.1 | 0.99 | 0.026 | 0.011 |
| H₂WO₄ | .025 | 89.85 | 71.45 | 55.55 | 0.777 | 0.081 |
| H₃O₄₄W₁₂P | | 84.47 | 67.52 | 46.90 | 0.692 | 0.074 |
| Mo₁₂O₄₀P | | 33.16 | 36.67 | 1.34 | 0.036 | 0.017 |

TABLE 4
Performance of tungsten acids for glycerol dehydration at 320° C.

| | Con- | | | | | |
|---|---|---|---|---|---|---|
| Catalyst | tact | Carbon | | Acrolein | | Acetol |
| (20 wt % on | Time | Balance | Conv. | Yield | Acrolein | Selec- |
| silica) | (sec) | (%) | (%) | (%) | Selectivity | tivity |
| H₂WO₄ | .013 | 93.72 | 50.16 | 38.61 | .77 | 0.105 |
| H₃O₄₄W₁₂P | | 95.95 | 31.85 | 24.37 | .783 | 0.111 |
| H₂WO₄ | .024 | 91.57 | 63.09 | 47.94 | .76 | 0.107 |
| H₃O₄₄W₁₂P | | 87.48 | 54.58 | 36.93 | .665 | 0.091 |

At 300° C., both tungsten- and molybdenum-based acids can be active, with tungsten acids showing acrolein selectivities in the range of 0.69-0.81. At shorter contact times selectivities can increase. Similar trends can be observed for experiments conducted at 320° C., though overall selectivities can be lower at this temperature.

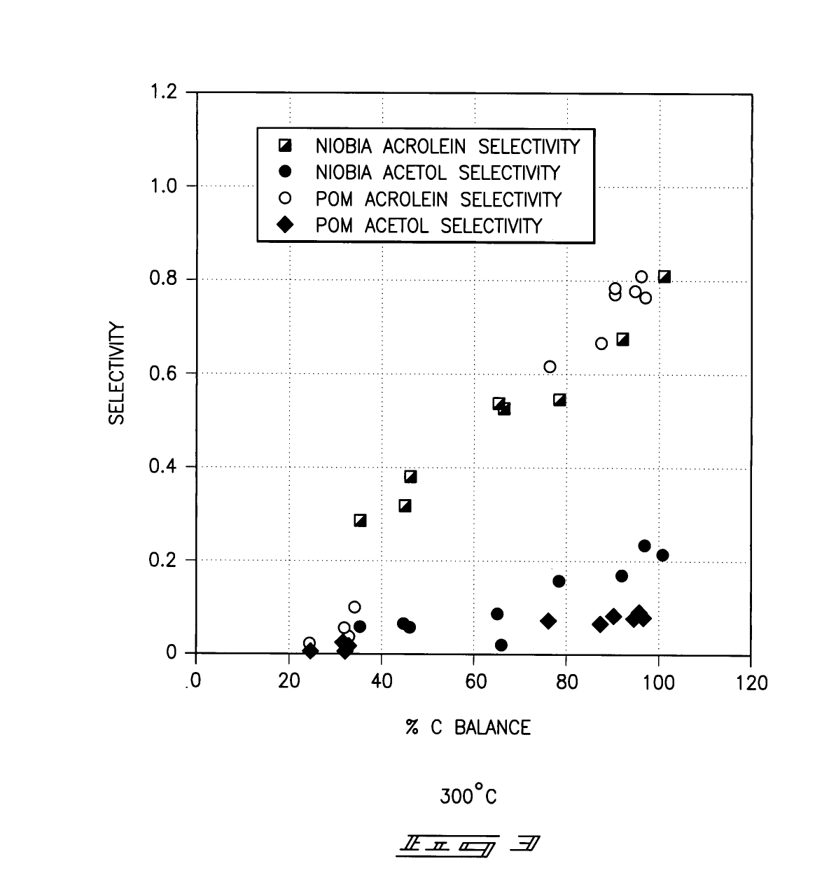

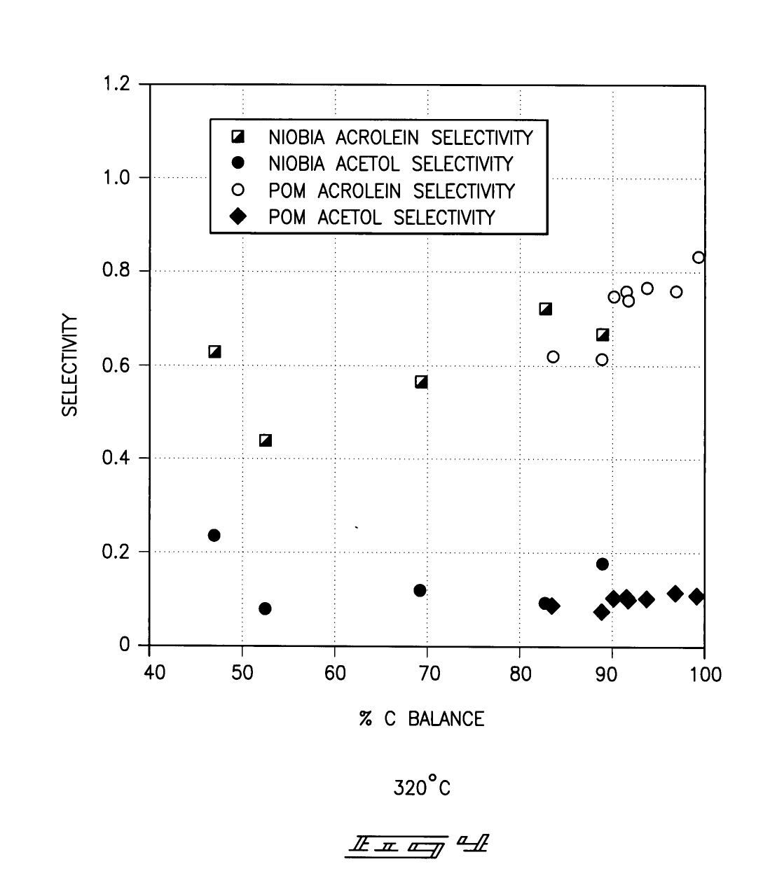

Plots of selectivity versus carbon balance for example production processes are shown in FIGS. 3 and 4. That the acrolein selectivity-carbon balance curves for both niobates and tungstates fall on the same line may indicate that both share similar propensities for acrolein decomposition or polymerization. In contrast, the two appear to differ in their relation to acetol selectivity possibly indicating that the acetol decomposition is more sensitive to overall acid strength.

Catalyst compositions incorporating phosphoric acid, metal phosphates and niobia were prepared and screened for glycerol dehydration activity. Results are presented in Table 5 below.

TABLE 5
Performance of mixed catalysts for glycerol dehydration at 300° C.

| | Contact | Carbon | | Acrolein | | |
|---|---|---|---|---|---|---|
| | Time | Balance | Conv. | Yield | Acrolein | Acetol |
| Catalyst (on SiO₂) | (sec) | (%) | (%) | (%) | Selectivity | Selectivity |
| 5% Mn/Nb₂O₅ | 0.15 | 94.66 | 16.78 | 9 | 0.536 | 0.144 |
| 79% (5% Mn/Nb₂O₅) | | 99.72 | 12.72 | 9.61 | 0.769 | 0.223 |
| 5% Co/Nb₂O₅ | | 87.01 | 25.69 | 10.17 | 0.4 | 0.1 |
| 10% H₃PO₄/Nb₂O₅ | | 66.53 | 53.94 | 13.76 | 0.258 | 0.127 |
| 20% H₃PO₄/Nb₂O₅ | | 90.07 | 50.04 | 33.41 | 0.672 | 0.135 |
| 2.9% Mn77% Nb₂O₅ 8% H₃PO₄ | | 100.65 | 12.29 | 10.25 | 0.842 | 0.219 |
| Mn/H₃PO₄/Nb₂O₅ | | 90.49 | 21.81 | 9.9 | 0.461 | 0.113 |
| 79% (5% Mn/Nb₂O₅) | 0.25 | 63.04 | 61.85 | 19.3 | 0.309 | 0.093 |
| 10% H₃PO₄/Nb₂O₅ | | 48.35 | 90.81 | 26.67 | 0.295 | 0.138 |
| 20% H₃PO₄/Nb₂O₅ | | 84.53 | 75.31 | 49.74 | 0.663 | 0.135 |
| 2.9% Mn77% Nb₂O₅ 8% H₃PO₄ | | 90.04 | 41.72 | 25.29 | 0.607 | 0.156 |
| Mn/H₃PO₄/Nb₂O₅ | | 86.17 | 33.8 | 16.07 | 0.485 | 0.119 |

In compliance with the statute, this disclosure has been provided in language more or less specific as to structural and methodical features. It is to be understood, however, that the disclosure is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A chemical production process comprising exposing a reactant composition to a catalyst composition to form a product composition, wherein:
the reactant composition comprises a multihydric alcohol compound;
the catalyst composition comprises one or more elements of groups 5 and 6 of the periodic table of elements; and
the product composition comprises a carbonyl compound.

2. The chemical production process of claim 1 wherein:
the multihydric alcohol compound is glycerol; and
the product composition comprises one or both of acrolein and acetol.

3. The chemical production process of claim 1 wherein at least a portion of the product composition is later utilized as a reactant.

4. The chemical production process of claim 1 wherein the catalyst composition comprises one or more of Nb, Mo, and W.

5. The chemical production process of claim 1 wherein the catalyst composition comprises hydrated Nb.

6. The chemical production process of claim 1 wherein the catalyst composition comprises one or both of tungstic acid and phosphotungstic acid.

7. The chemical production process of claim 1 wherein the catalyst composition comprises phosphomolybdic acid.

8. The chemical production process of claim 1 wherein the catalyst composition is supported.

9. The chemical production process of claim 1 wherein the catalyst composition is supported with a silica support.

10. The chemical production process of claim 1 wherein the multihydric alcohol compound is glycerol and the product composition comprises both acrolein and acetol.

11. The chemical production process of claim 10 wherein every mole of glycerol exposed to the catalyst composition forms at least about 0.1 moles of product composition.

12. The chemical production process of claim 10 wherein every mole of glycerol exposed to the catalyst composition forms about 0.1 moles to about 0.99 moles of product composition.

13. The chemical production process of claim 10 wherein a ratio of acrolein to acetol is at least about 3:2.

14. The chemical production process of claim 10 wherein a ratio of acrolein to acetol is from about 3:2 to about 10:1.

15. The chemical production process of claim 1 wherein during the exposing, the catalyst composition is maintained at a temperature of from about 200° C. to about 500° C.

16. The chemical production process of claim 1 wherein during the exposing, the catalyst composition is maintained at a temperature of from about 280° C. to 320° C.

17. A dehydration catalyst composition comprising one or more of niobia, hydrated niobia, tungstic acid, phosphotungstic acid, and phosphomolybdic acid.

18. The catalyst composition of claim 17 further comprising a solid support material.

19. The catalyst composition of claim 18 wherein the solid support material comprises Si.

20. A chemical production system comprising a reactant reservoir coupled to a reactor, the reactor containing a catalyst comprising one or more elements of groups 5 and 6 of the periodic table of elements.

21. The system of claim 20 wherein the catalyst composition comprises one or more of Nb, Mo, and W.

22. The system of claim 20 wherein the catalyst composition comprises hydrated Nb.

23. The system of claim 20 wherein the catalyst composition comprises one or both of tungstic acid and phosphotungstic acid.

24. The system of claim 20 wherein the catalyst composition comprises phosphomolybdic acid.

25. The system of claim 20 wherein the catalyst composition is supported.

26. The system of claim 20 wherein the catalyst composition is supported with a silica support.
